(19) United States
(12) Patent Application Publication
(10) Pub. No.: US 2018/0110643 A1
(43) Pub. Date: Apr. 26, 2018

(54) Hinged Wrist and Hand Support

(71) Applicant: Curtis Bryan Carlson, Holland, NE (US)

(72) Inventor: Curtis Bryan Carlson, Holland, NE (US)

(21) Appl. No.: 15/791,060

(22) Filed: Oct. 23, 2017

Related U.S. Application Data
(60) Provisional application No. 62/496,593, filed on Oct. 24, 2016.

(51) Int. Cl. A61F 5/01; A61F 5/058

(52) U.S. Cl. CPC A61F 5/013; A61F 2005/0172; A61F 2005/0158; A61F 5/05866

(57) ABSTRACT

A bilateral rigid orthopedic support with pliable tabs that stabilize the wrist and hand, utilizing an adjustable hinge with a locking mechanism, providing the user the ability to lock the wrist into varying degrees of flexion and extension while simultaneously allowing for maximum support, with the addition of an orthopedic circumferential wrist and hand sleeve is disclosed. The combination of orthopedic sleeve materials and the rigid frame provide the required stability and compression while allowing for full phalange function.

### CLAIMING THE PRIORITY OF AN EARLIER APPLICATION

Provisional Application No. 62/495,593

Titled OM-3916 Wrist Support

Filed on Oct. 24, 2016

### FIELD OF THE INVENTION

This present invention relates to the field of orthopedic bracing and more specifically to wrist and hand supports.

### BACKGROUND OF THE INVENTION

The medical orthosis industry can be improved by providing a rigid wrist and hand support solution with a hinge with a locking mechanism that would provide the user the ability to lock the wrist into a variety of positions for the purpose of providing stability while allowing appropriate flexion and extension. In particular, what is needed is a rigid brace that is not overly rigid, bulky or heavy, that maintains joint stability yet permits a safe adjustable range of flexion and extension that will allow desired extension and flexion positioning. Additional improvement is needed in providing a two-in-one rigid wrist and hand support that combines a rigid brace frame with a hand and wrist compression sleeve, providing for the needs of stability and compression simultaneously while allowing the user full phalange function.

### BRIEF DESCRIPTIONS OF THE DRAWINGS

Further advantages of the disclosure are referenced in the detailed description when considered in unison with the figures, which indicate several elements displayed by several views.

FIG. 1 Is a side view of the of the hinged wrist and hand support with a wrist in a position of extension.

FIG. 2 Is a plan view of the distal frame containing the pliable distal tabs of the hinged wrist and hand support.

FIG. 3 Is a plan view of the proximal frame containing the pliable proximal tabs of the hinged wrist and hand support.

FIG. 4 Is a plan view of the distal frame containing the pliable distal tabs highlighting the interlocking washers of the hinged wrist and hand support.

FIG. 5 Is a plan view of the proximal frame containing the pliable proximal tabs, highlighting the proximal stay of hinged wrist and hand support.

FIG. 6. Is an exploded view of a hinge and locking mechanism of the hinged wrist and hand support.

FIG. 7 Is an exploded view of another embodiment of the hinge and locking mechanism of the hinged wrist and hand support.

FIG. 8. Is an assembly plan view of the hinge and locking mechanism of the hinged wrist and hand support.

FIG. 9 Is an assembly plan view of another embodiment of the hinge and locking mechanism of the hinged wrist and hand support.

FIG. 10. Is an assembly plan view of the hinge and locking mechanism connecting the distal and proximal frames of the hinged wrist and hand support.

FIG. 11. Is an assembly plan view of another embodiment of the hinge and locking mechanism connecting the distal and proximal frames of the hinged wrist and hand support.

FIG. 12 Is a sectional view of the circumferential wrist and hand sleeve of the hinged wrist and hand support.

FIG. 13 Is a sectional view of the hand and wrist support without the circumferential wrist and hand sleeve.

FIG. 14 Is a plan view of the dorsal side of the bilateral hinged wrist and hand support showing the distal strap, attached by a rivet providing 360° rotation of the strap, wrapped around the hand and worn on the left hand.

FIG. 15 Is a plan view of the dorsal side of the bilateral hinged wrist and hand support showing the distal strap, attached by a rivet providing 360° rotation of the strap, wrapped around the wrist and worn on the right hand.

FIG. 16 Is a side view of the of the hinged wrist and hand support with a wrist locked into a position of extension.

FIG. 17 Is a side view of the of the hinged wrist and hand support with a wrist locked into a position of flexion.

### BRIEF SUMMARY OF THE INVENTION

This disclosure is a bilateral wrist and hand support with a hinge and locking mechanism as well as a circumferential wrist and hand sleeve. The hinged wrist and hand support can be used to assist in the treatment of carpal tunnel syndrome, osteoarthritis, rheumatoid arthritis, trigger finger, navicular fracture bone of wrist, fracture lower end of radius for closed fracture, tendonitis/tenosynovitis of wrist and/or hand, fracture of the metacarpal bone, sprain of wrist, idiopathic avascular necrosis radius, or pain in the forearm, wrist, or hand as well as other indications.

The wrist and hand support includes a rigid brace frame made of aluminum having several pliable outwardly extending tabs along the rigid proximal brace frame. The distal set of pliable outwardly extending tabs wrap around the dorsal and palmer side of the hand. The proximal set of pliable outwardly extending tabs wrap around the ventral and dorsal side of the forearm to provide support.

The hinge and locking mechanism is designed to line up with the wrist joint. The hinge allows the user to adjust the brace into a variety of positions of extension and flexion and lock the brace into a desired position. The hinge and locking mechanism consists of a screw with a dial head, stopping element, two interlocking washers and a groove nut working together to interlock when tightened, securing the proximal section of the frame to the distal section, holding the wrist in the desired position. The interlocking washers are attached to the brace frame by a set stays, a rivet attaches the stay to the brace frame so that it cannot be detached from the frame. The hinge with a locking mechanism has a stopping element at the end that prohibits it from being unscrewed completely from the grooved nut, eliminating the problem of the hinge with a locking mechanism coming apart. Polyester foam, cut into the shape of the brace frame, is inserted and covered with an external polyester mesh fabric to protect the skin from irritation from the hinge with a locking mechanism rubbing against the wrist. The wrist and hand support allows for full phalange function.

A lightweight lycra circumferential compression sleeve is affixed inside the brace frame with the purpose of increasing compression and comfort, while reducing swelling. The wrist and hand support combines the advantages of compression with the secure fit and comfortable feel of a lycra and nylon compression sleeve supported by a rigid brace frame with pliable tabs allowing the wrist and hand support to be simultaneously comfortable, pliable and supportive.

Nylon straps containing a hook and loop fastening system, used to tighten the brace around the forearm, wrist, and hand, are sewn onto the external polyester mesh brace material and tethered by a d-ring, providing maximum support and comfort. The distal strap is attached with a rivet providing a 360° rotation of the strap allowing for a variety of configurations.

### DETAILED DESCRIPTION

A better understanding of the different embodiments and uses of this disclosure may be gained from the following description read in conjunction with the attached drawings and numeric references referring to the various elements.

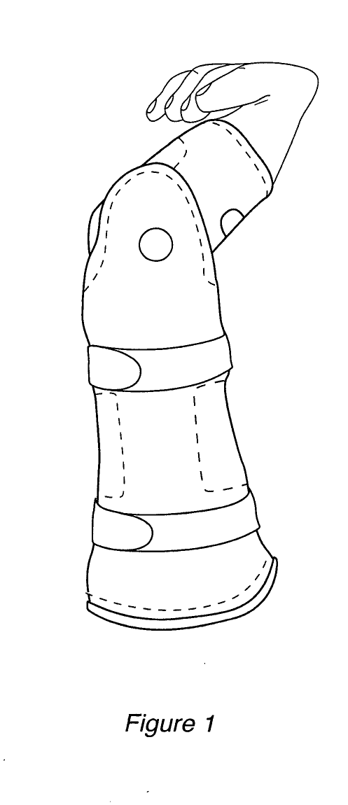

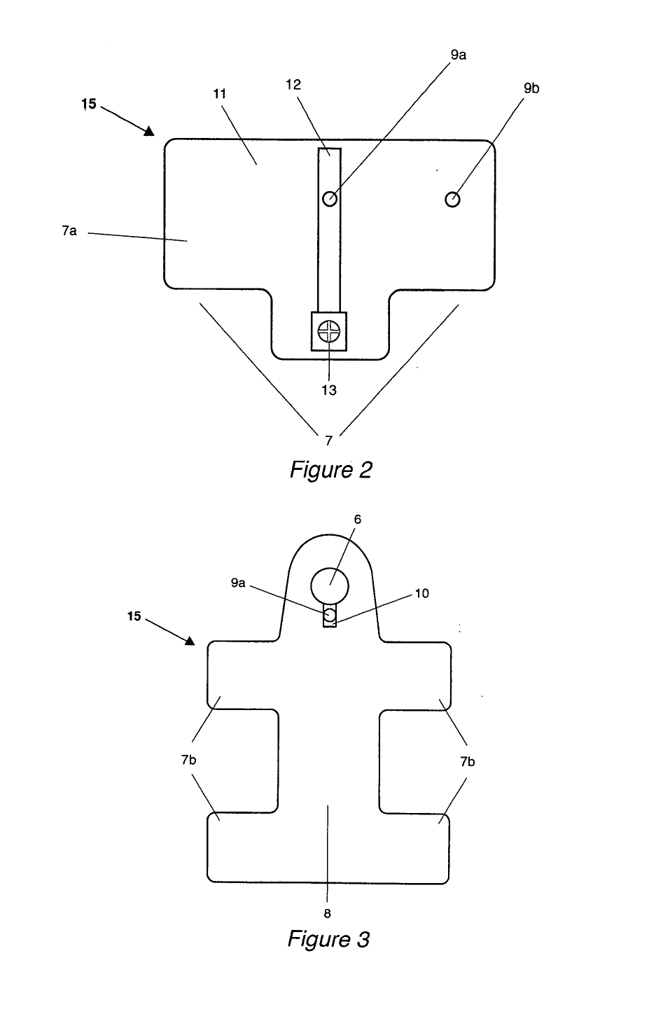

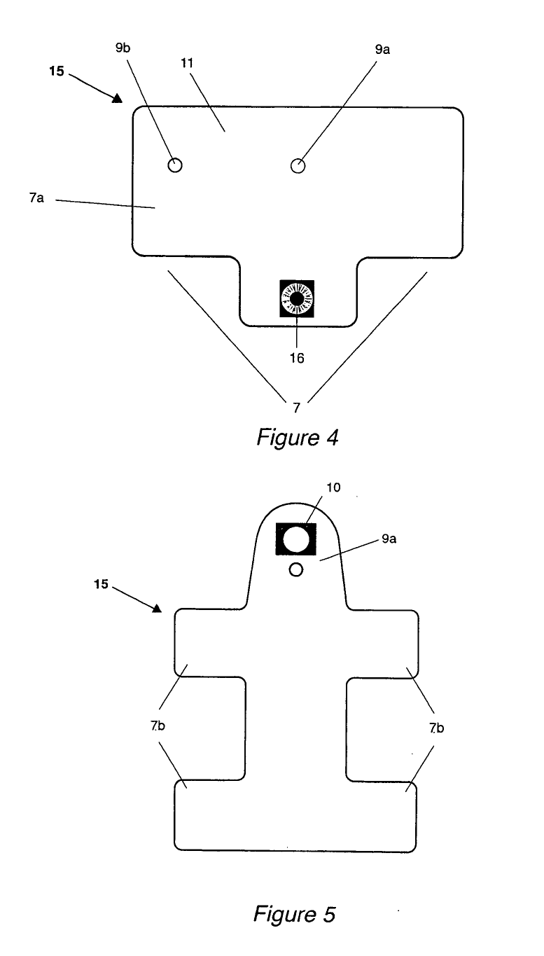

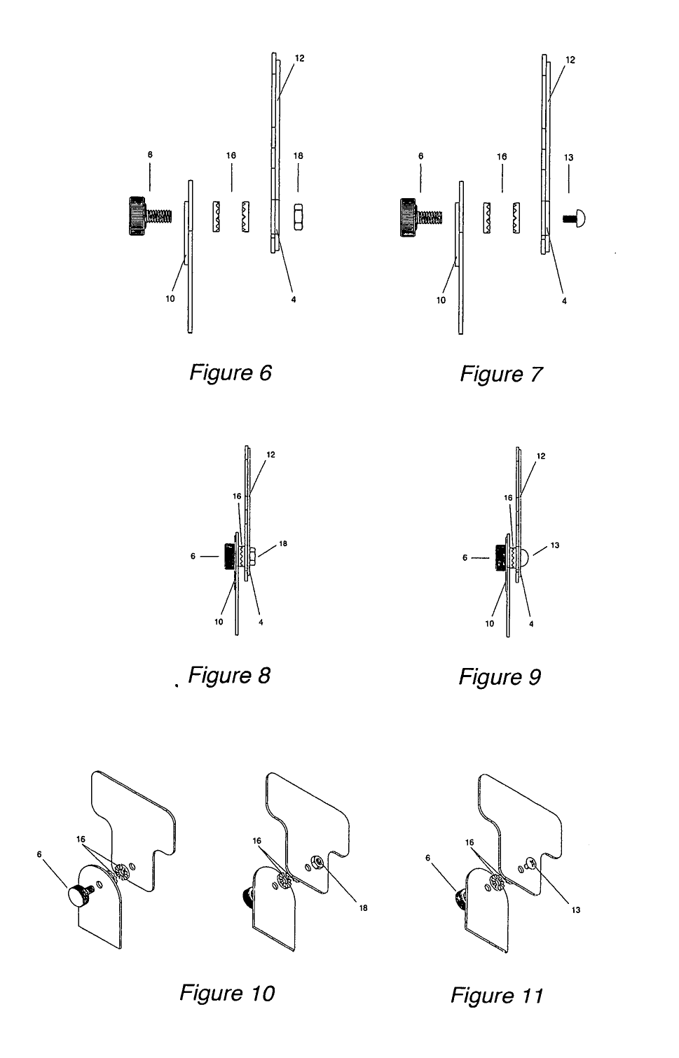

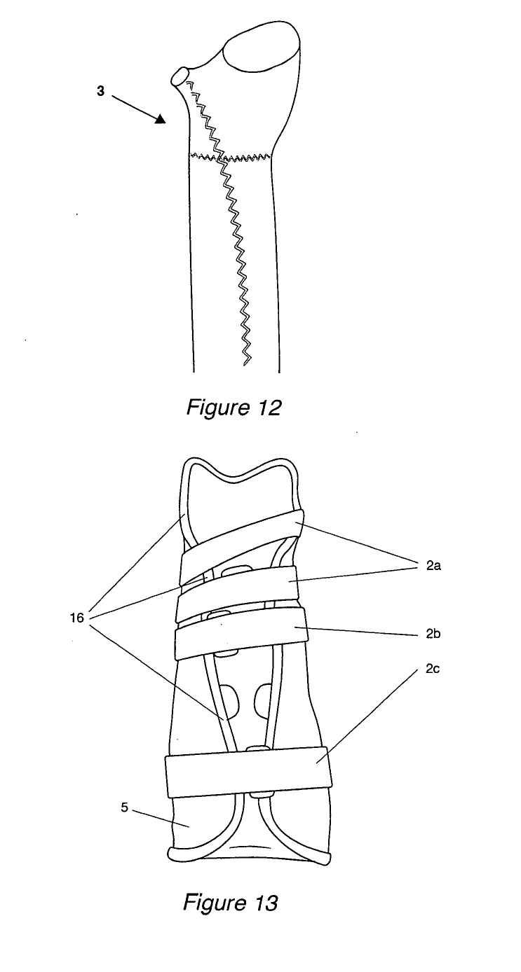

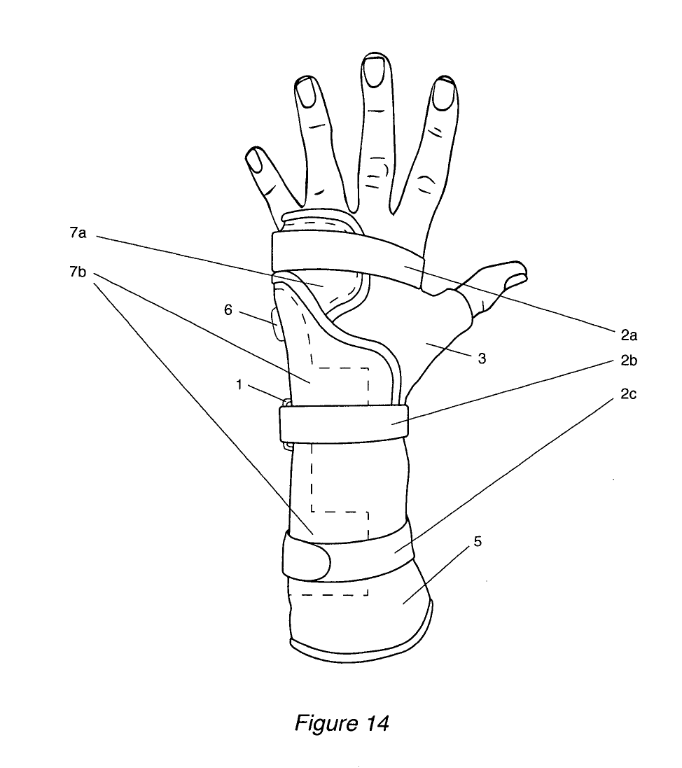

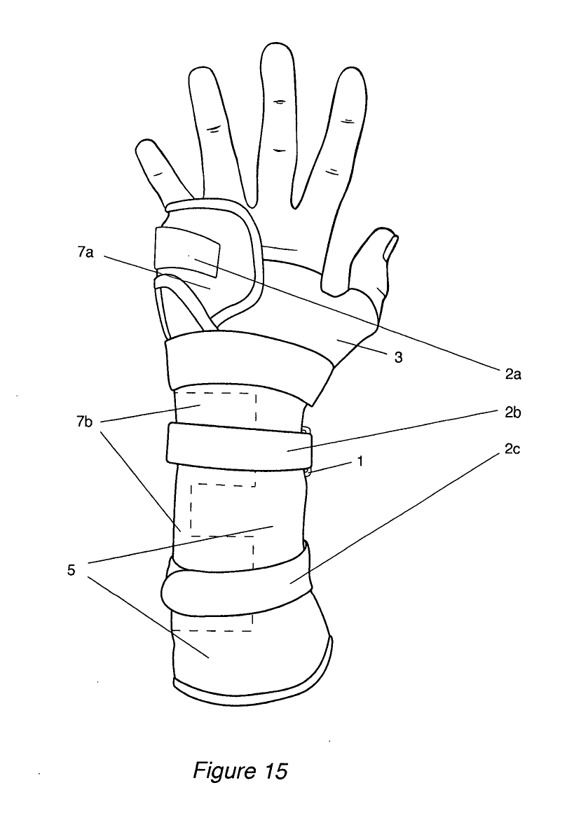

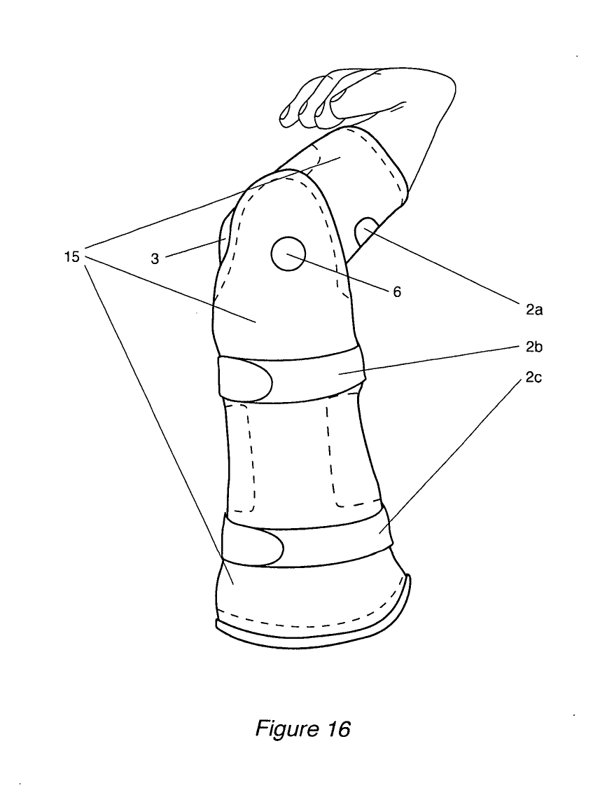

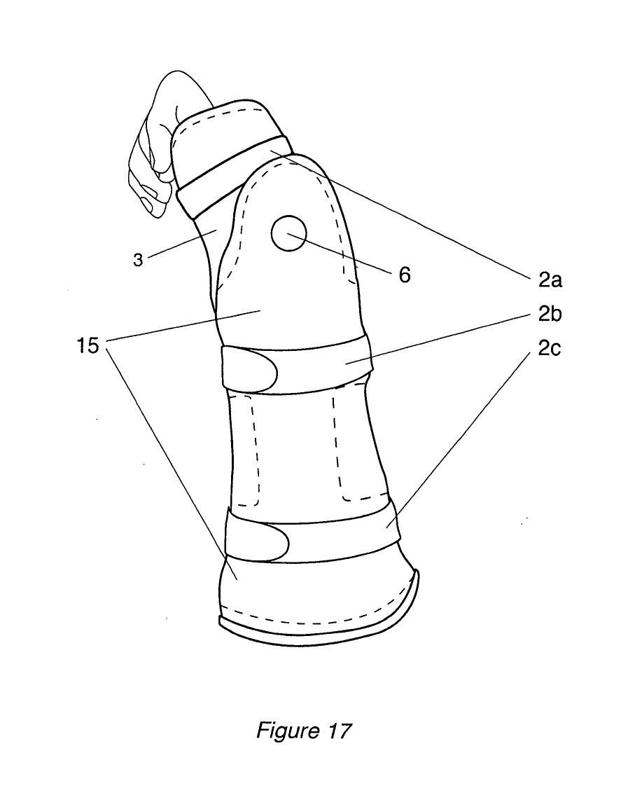

With reference to FIGS. 1-17 one embodiment shown is a wrist and hand support FIG. 1 having a rigid brace frame with pliable tabs FIGS. 2, 3, 4 & 5 a hinge with a locking mechanism FIG. 6-11 for adjusting the flexion and extension of the wrist FIGS. 16 & 17 while providing compression through a wrist and hand sleeve FIG. 12 able to be worn bilaterally FIGS. 14 & 15.

The rigid aluminum bilateral brace frame 15 consists of pliable distal tabs 7a, pliable proximal tabs 7b, a distal frame 11, FIGS. 2 & 4 and proximal frame 8, FIG. 3. The brace frame 15 maintains its shape after forming the pliable distal tabs 7a and pliable proximal tabs 7b to the forearm, wrist and hand of the user, providing a protective shield as well as the required amount of stabilization. Another material that could be used to make the rigid frame 15 with pliable distal tabs 7a & pliable proximal tabs 7b include polypropylene thermoplastic polymer.

The rigid aluminum distal frame 11, FIGS. 2 & 4 houses the pliable distal tabs 7a extending outward and wrap around the dorsal and palmer side of the hand FIGS. 14 & 15. Wherein the proximal frame 8 houses the pliable proximal tabs of extending outward 7b which wrap around the ventral and dorsal side of the forearm to provide stabilization FIGS. 14 & 15.

The distal frame 11, FIG. 2 also houses a distal stay 12 attached to the aluminum distal frame 11 through a rivet 9a. The distal frame 11 is connected to the proximal frame 8, by a hinge with a locking mechanism FIG. 6-11.

One embodiment of the hinge and locking mechanism FIGS. 6, 8, & 10 consists of a screw with a dial head 6, stop nut 18, two interlocking washers 16 and a groove nut 4 working together to interlock when tightened, passing through a proximal stay 10 and the distal stay 12, securing the proximal frame 8 to the distal frame 11, securing the wrist in the desired position of extension or flexion FIG. 16-17. The interlocking washers 16 are attached to the brace frame 15 by the distal stay 12 and the proximal stay 10. A rivet 9a attaches the distal stay 12 to the distal frame 11. The screw with a dial head 6 has a stopping nut 18 at the end that prohibits it from being unscrewed completely from the grooved nut 4, eliminating the problem of the hinge and locking mechanism coming apart.

Another embodiment of the hinge and locking mechanism FIGS. 7, 9 & 11 could include a screw with a dial head 6, interlocking washers 16, a grooved nut 4 and a flat head stop screw 13 working together to interlock when the screw with a dial head 6 is tightened FIG. 9, passing through a proximal stay 10 and the distal stay 12 securing the proximal brace frame 8 to the distal frame 11, holding the wrist in the desired position of extension FIG. 16 or flexion FIG. 17. The grooved nut 4 is attached to the distal frame 11 by the distal stay 12 and the rivet 9a attaches the distal stay 12 onto the distal frame 11. The screw with dial head 6 has a stop screw 13 at the end that prohibits the screw with dial head 6 from being unscrewed completely from the grooved nut 4, eliminating the hinge and locking mechanism FIGS. 7, 9 & 11 from coming apart.

The locking mechanism FIG. 6-11 is engaged by turning, and therefore tightening, the screw dial head 6 maintaining the wrist in the desired position of extension FIG. 16 & flexion FIG. 17. The hinge with a locking mechanism FIG. 6-11 allows the distal frame 11 to move freely through the full range of motion and when locked provides support during extension FIG. 16 and flexion FIG. 17.

A strip of polyester foam, nylon, spandex and polyester mesh is cut into the exact shape of the brace frame 15, laid on the brace frame 15, held in its place by covering the entirety of the brace frame 15 with polyester mesh material 5 and sewn together creating a binding 16. This forms the brace support structure FIG. 13 and allows space for the shape of the users forearm by leaving a hollow cavity which will house the circumferential wrist and hand sleeve 3, FIG. 12.

A combination of lightweight elastic lycra and nylon are used to create the wrist and hand circumferential compression sleeve 3, FIG. 12. The wrist and hand sleeve 3 is then sewn into the interior brace material 5, FIG. 13 with the purpose of increasing compression and comfort, while reducing swelling. Two openings at the top of the wrist and hand sleeve 3 are cut to allow for full function of the fingers and thumb. Compression sleeve 3 materials could also include, but are not limited to, neoprene, latex, nylon and spandex or a combination thereof.

Three nylon straps 2a, 2b, & 2c are attached to the polyester mesh material 5 to tighten the brace FIGS. 14 & 15 to the wearer. The proximal straps 2b and 2c are sewn to the exterior polyester mesh brace material 5 and pass through a d-ring 1, made of polyoxymethylene, also attached to the polyester mesh material 5, and house a hook and loop fastener system used to tighten the brace around the forearm, wrist, and hand providing maximum support and comfort. Fasteners could also include, but are not limited to, closure systems produced by Boa Technology illustrated in U.S. Pat. No. 8,277,401. The distal strap 2a, is attached by a rivet 9b with 360° rotation capability. The distal strap 2a can be tightened using a variety of strapping methods to achieve the desired fit. FIG. 14 illustrates the distal strap 2a, attached by the 360° rivet 9b, wrapped around the hand. FIG. 15 illustrates the hinged wrist and hand support showing the distal strap 2a, attached by the 360° rivet 9b, wrapped around the wrist.

The bilateral hand and wrist support was created to fit most-to-all users by creating a variety of sizes. Other embodiments would include additional sizes as well as a universal, one-size-fits-all embodiment.

This disclosure is a needed addition to the field of orthopedic wrist and hand supports. A need exists for a pliable bilateral orthopedic wrist and hand support that incorporates a compression sleeve for swelling as well as an adjustable hinge with locking mechanism, providing the user the ability to lock the wrist into a variety of extension and flexion positions configured to cooperate with the wrist joint and allowing for full phalange function. This support can be used to assist in the treatment of carpal tunnel syndrome, osteoarthritis, rheumatoid arthritis, trigger finger, navicular fracture bone of wrist, fracture lower end of radius for closed fracture, tendonitis/tenosynovitis of wrist and/or hand, fracture of the metacarpal bone, sprain of wrist, idiopathic avascular necrosis radius, or pain in the forearm, wrist, or hand as well as other indications.

I claim:

1. A device for supporting the wrist and hand comprising of:
a hinge with a locking mechanism which connects to
a two-piece rigid brace frame with pliable tabs which is housed in the external brace material which is attached to
a lightweight circumferential compression hand and wrist sleeve tightened by a closure system.

2. A device for supporting the wrist and hand having a hinge and locking mechanism comprising of:
a screw with a dial head which passes through the
a proximal stay,
a proximal frame,
two interlocking washers,
a distal frame,
a distal stay, which houses the grooved nut, and is completed by a stopping element, all working together to interlock when tightened, securing the proximal frame to the distal frame and securing the wrist and hand in a desired position.

3. A device for supporting the wrist and hand comprising of:
a hand and wrist circumferential compression sleeve which attaches to an interior brace material that lines the rigid brace frame.

4. A device for supporting the wrist and hand, according to claim 1, wherein the rigid brace frame including the tabs is made of pliable material.

5. A device for supporting the wrist and hand, according to claim 1, wherein the frame is covered by a combination of polyester foam, nylon, spandex and polyester mesh.

6. A device for supporting the wrist and hand, according to claim 1, is tightened by a closure system used to secure the brace around the forearm, hand and wrist.

7. A device for supporting the wrist and hand, according to claim 3, wherein the circumferential wrist and hand sleeve is made of a combination of elastic lycra and nylon.
